(19) United States
(12) Patent Application Publication
(10) Pub. No.: US 2007/0186934 A1
(43) Pub. Date: Aug. 16, 2007

(54) Tracheostomy device

(76) Inventor: James T. DeLuca, Forest Hills Gardens, NY (US)

Correspondence Address: Charles E. Temko, Esq., 22 Marion Road, Westport, CT, 06880, US

(21) Appl. No.: 11/355,142

(22) Filed: Feb. 16, 2006

(51) Int. Cl. A61B 17/34; A62B 9/06; A61M 16/00

(52) U.S. Cl. 128/207.29; 128/207.14; 606/185

(57) ABSTRACT

A device for creating a flattened opening in the neck of a victim for temporary stabilization by creating an air passage for breathing. The device includes a planar knife element and a surrounding flattened tube which is inserted simultaneously to form an air passage after removal of the knife element. The tube element includes laterally-extending flange means for limiting the degree of penetration during installation. In a preferred embodiment, the tube element includes laterally-extending structure for engaging the ends of the created air passage to retain the installed device in position.

### RELATED APPLICATION

Reference is made to my copending provisional application Ser. No. 60/651,406 filed Feb. 28, 2005, to which a claim of priority is made.

### BACKGROUND OF THE INVENTION

This invention relates generally to the field of medical devices, and more particularly to an improved tracheotomy device.

Devices of this general type are known in the art and are often useful during emergency situations where a patient experiences blockage in breathing due to the presence of a lodged mass or as an accidental occurrence, and the like. In many situations, unless substantially immediate relief is provided, the patient may suffocate.

In such situations, it is necessary to perform emergency surgery to provide a temporary opening in the trachea through which the patient can breathe. Unless this is performed carefully, it may result in an opening which is not adequately large, and tissues may be damaged in a manner which prevents or hinders subsequent healing. In normal operating room procedures, it is known to provide openings using a needle and subsequent insertion of a flexible tube to provide injection and removal of various fluids. However, in emergency situations, in most cases, there is no useful tool for providing a temporary breathing channel.

### SUMMARY OF THE INVENTION

Briefly stated, the invention contemplates the provision of a portable device which may be used by persons having less than full surgical skills to provide an opening located between a pair of adjacent cartilaginous rings located at the lower portion of the neck of the patient to provide an opening through which a relatively flat tube may be inserted to provide a continuous air passage to permit the victim to breathe until he is transported to a medical facility. To this end, the disclosed device includes a blade of planar configuration having an inner sharpened edge, the blade being surrounded by a flattened tube which is inserted into the opening formed by the blade, following which the blade is withdrawn so that the tube forms a passage for breathing. The blade is part of an element which enables the user to control the initial location of the blade and apply the proper degree of pressure to the same for penetration. The tube preferably includes an enlargement at an outer end thereof to maintain position and permit easy removal. In a second disclosed embodiment, the device includes a pair of oppositely disposed resilient members on the tube which maintain the tube in proper position after insertion in the created opening in the neck of the patient to cooperate with the enlargement at the outer end of the tube.

### BRIEF DESCRIPTION OF THE DRAWINGS

In the drawings, to which reference will be made in the specification, similar reference characters have been employed to designate corresponding parts throughout the several views.

FIG. 1 is a side elevational view of a first embodiment of the invention.

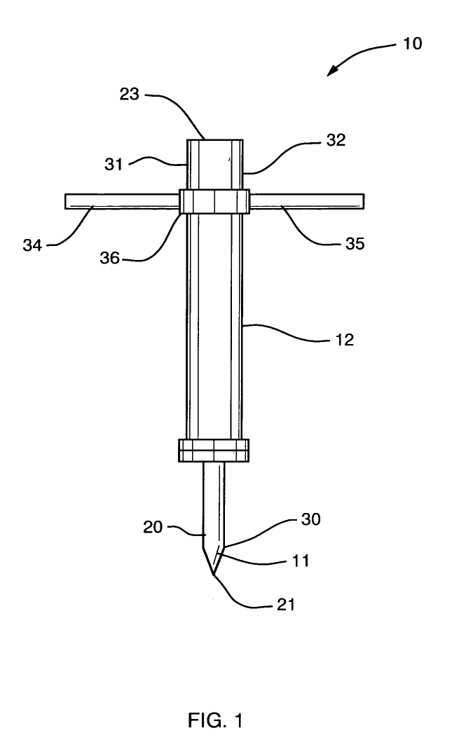

FIG. 2 is a perspective view of a second embodiment of the invention.

FIG. 3 is a side embodiment of a blade element which comprises a part of the second embodiment.

### DETAILED DESCRIPTION OF THE DISCLOSED EMBODIMENTS

In accordance with the first embodiment of the invention, the device, generally indicated by reference character 10, comprises broadly, a knife element 11, and a tube element 12.

The knife element 11 may be made of metal or synthetic resinous materials, and includes a planar blade 20 having a first transversely-extending sharpened edge 21. A manually engageable outer end 32 includes a flattened surface 23 for engagement by the fingers of the user, so that once the device is positioned against the lower portion of the neck, penetration can be adequately controlled with minimum tissue damage. These members may be arranged for pivotal movement to provide convenient storage.

The tube element 12 is preferably formed from metallic tubing. When the device is assembled, an inner or first end 30 is positioned adjacent the cutting edge of the knife element, while an outer end 31 may be provided with an enlargement 36 to position the tube within the formed opening when the knife element is subsequently withdrawn.

The device will normally be stored in assembled condition, ready for immediate use, preferably within a sterile container or wrapping (not shown). Upon the occurrence of an emergency, the cartilaginous rings in the lower portion of the neck are manually located, following which the knife end is employed to penetrate between them using controlled pressure exerted at the outer end of the blade element. The tube element 12 simultaneously enters the opening and remains in position while the knife element is disconnected therefrom to provide a relatively flat passage for breathing to stabilize the patient prior to transportation to a medical facility where the initial breathing problem is corrected. At this time, the tube element may be removed and necessary suturing of the cut tissue performed. Since the configuration and size of the opening in tissue is predetermined, penetration of the tube element is limited to the degree necessary to establish air communication. The device may be formed in varying sizes to be used depending upon the size of the neck of the user.

Turning now to the second embodiment of the invention, the device, generally indicated by reference character 110 differs from the first embodiment in that the principal components are of molded or cast construction which facilitates manufacture as well as improved ease in use. It also includes improved means for mounting the tube element in installed condition.

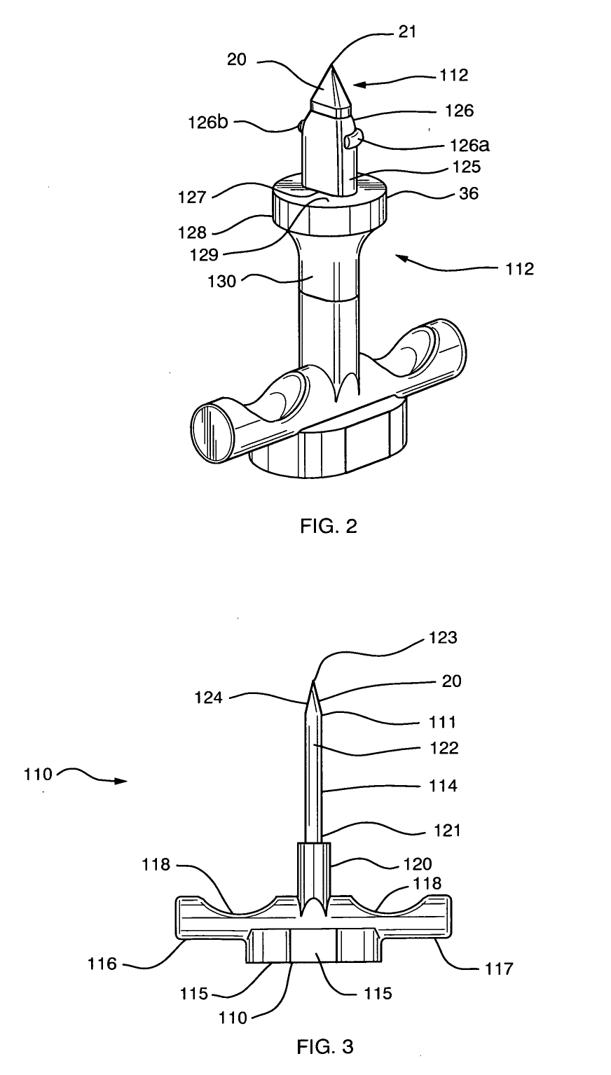

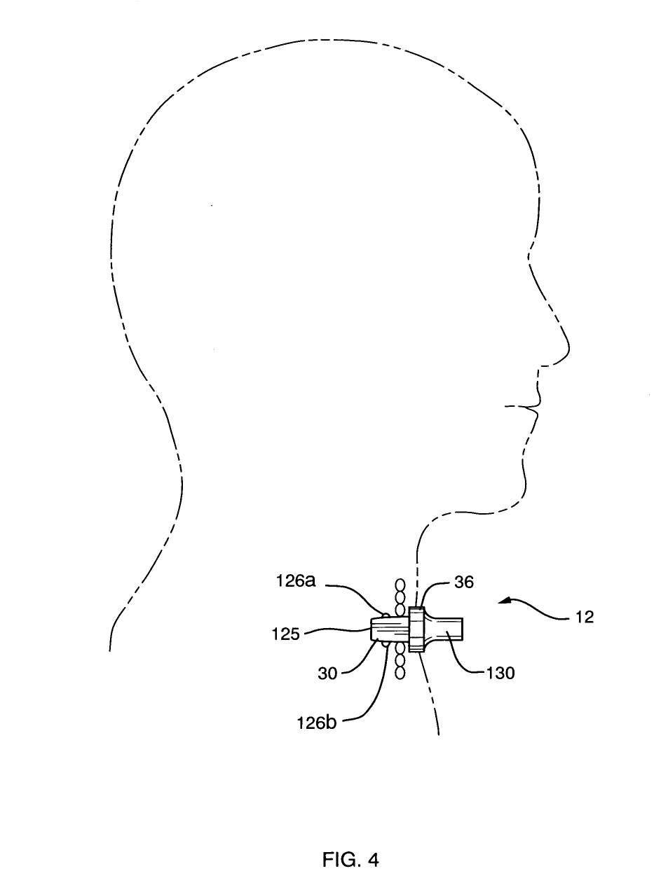

Referring to FIGS. 2 and 3, the device comprises a knife element 111 and a tube element 112.

The knife element 111 includes a manually-engageable base member 113 of metal or synthetic resinous material, and an elongated knife member 114, preferably of metallic material. The base member 113 includes a terminal portion 115 which enables the device to be supported on a horizontal surface to assist use. A pair of aligned finger-engaging members 116 and 117 extend laterally and include recesses 118. The planar surface 119 is engaged by the thumb of the user. A hollow shaft 120 includes a socket (not shown) for reception of a first end 121 of knife blade 122. A second end 123 is of pointed configuration and tapers to a generally tapered cross section at 124.

The tube element 112 is configured to provide means for maintaining itself in installed condition and provide a manually-engageable surface for convenient disengagement from the patient when no longer needed. As best understood from a consideration of FIG. 2, the tube element includes a generally planar member 125 having a tapered end 126 and laterally projecting retaining members 126a and 126b which engage the ends of the opening in the neck of the patient. An outer end 127 interconnects with an annular flange 128 (FIG. 3) having a surface 129 which abuts the area surrounding the planar member 125. A projecting flattened member 130 permits manual engagement for removal of the device when no longer needed.

I wish it to be understood that I do not consider the invention to be limited to the details of structure shown and described in the specification, for obvious modifications will occur to those skilled in the art to which the invention pertains.

I claim:

1. A tracheotomy device for creating a breathing passage in the lower neck portion of a victim, comprising: a generally planar knife element having a cutting edge at a first end thereof and a manually-engageable second end; and a flattened flexible tube element surrounding said knife element including a first end positioned adjacent said first end of said knife element, whereby said device may be employed to penetrate the neck of a victim between a pair of adjacent cartilaginous members to create a flattened opening engaged by said tube element upon axially-directed removal of said knife element.

2. A device in accordance with claim 1, further comprising an enlargement on a second end of said tube element to position said tube element after installation is fixed relative to said opening.

3. A device in accordance with claim 2, further comprising at least one laterally-projecting member for engaging the ends of an elongated opening created by said knife element upon installation.

4. A device in accordance with claim 2, said knife element having an outer end thereof including laterally-extending members for engagement by fingers of a user, and an outer terminal member for engagement of the thumb of said user to facilitate insertion of said knife element to form an opening in the neck of a patient.

5. A device in accordance with claim 2, said knife element including a laterally-extending flanged member for determining the degree of penetration of said knife element.
